(19) United States
(12) Patent Application Publication
(10) Pub. No.: US 2010/0040660 A1
(43) Pub. Date: Feb. 18, 2010

(54) DEVELOPMENT OF A TISSUE - ENGINEERED SCAFFOLD FOR NERVE REGENERATION USING A BIOCOMPATIBLE AND INJECTABLE HYDROGEL

(75) Inventors: Moon Suk Kim, Seoul (KR); Hai Bang Lee, Seoul (KR); Ju Young Lee, Seoul (KR); Hyun Hee Ahn, Seoul (KR); Jung Hwa Lee, Seoul (KR); Kyung Sook Kim, Seoul (KR)

Correspondence Address: THE NATH LAW GROUP, 112 South West Street, Alexandria, VA, 22314, US

(73) Assignee: KOREA RESEARCH INSTITUTE OF CHEMICAL TECHNOLOGY, Daejeon (KR)

(21) Appl. No.: 12/189,951

(22) Filed: Aug. 12, 2008

(51) Int. Cl. A61K 9/00; C12N 5/00; A61K 35/12; A61P 25/00

(52) U.S. Cl. 424/423; 435/396; 435/397; 424/93.7

(57) ABSTRACT

The present invention relates to a tissue-engineered scaffold prepared by using a biocompatible and injectable hydrogel, and particularly to a tissue-engineered scaffold capable of regenerating or recovering an injured spinal nerve for central nervous system after being implanted to connect neurons, prepared by combining an adult stem cell or a nerve cell with a physiologically active material on tissue-engineered carriers comprising biocompatible and temperature-sensitive polyethylene glycol/polyester block copolymer or biocompatible and injectable hydrogel made of small intestinal submucosa tissue powder with sol-gel phase transition behavior.

### BACKGROUND

1. Technical Field

The present invention relates to a tissue-engineered scaffold prepared by using a biocompatible and injectable hydrogel. In particular, the present invention relates to a tissue-engineered scaffold capable of regenerating or recovering an injured spinal nerve for central nervous system after being implanted to connect neurons, prepared by combining an adult stem cell or a nerve cell with a physiologically active material on tissue-engineered carriers comprising a biocompatible and temperature-sensitive polyethylene glycol/polyester block copolymer or biocompatible and injectable hydrogel made of small intestinal submucosa tissue powder with sol-gel phase transition behavior.

2. Background Art

It has been known that nervous systems, especially central nervous system, may be impaired functionally once it is damaged and it is not impossible to regenerate the damaged nerves or recover from the nerve injury. The recent progress in neuroscience using molecular biology techniques, however, made it possible to elucidate the structures and functions of neurons at molecular level. Further, as the important roles of neurotransmitters and nerve growth factors have been emphasized for the past few decades, there have been an increasing number of evidences collected suggesting the central nervous system may be regenerated contrary to the long and old belief.

One way of regenerating the damaged nerve cells may be to provide a biological condition suitable for the recovery. Another way of treating the damaged nerve cells may be to increase the capacity of regeneration by strengthening the neurons themselves. The best known method to improve the biological condition at present is to exclude protein inhibitors secreted from gliacytes, which are known to prevent nerve regeneration, and at the same time introduce those cells which are known essential for nerve regeneration, such as Schwann cells, olfactory ensheathing cells and neural stem cells from the external environment. Besides, in strengthening the regeneration, it has been known very effective to administer neurotransmitters and nerve growth factors. The neurotransmitters such as NGF (nerve growth factor), BDNF (brain-derived neurotropic factor), NT-3 (neurotrophin-3) and NT-4/5 (neurotrophin-4/5) play a role to regulate the growth, differentiation, survival and death of neurons in the central nervous system. In detail, the nerve growth factor used for the treatment of neural disorders is known to be involved in regeneration of nerve tissues and cells and to stimulate their growth. Further, it has been proven effective to treat a spinal cord injury.

Stem cells can replace any kind of tissues or cells correctly at any location and differentiate into precursors of a specific cell. Thereby, the stem cells can become a very good cell source useful for regenerative therapy and may be used for treating and recovering an injured organ. The stem cells are broadly classified to an embryonic stem cell and an adult stem cell. The embryonic stem cell can be isolated from an early or fetal gonocyte. The adult stem cell can be isolated from bone marrow, adipose tissues and muscle tissues of an adult. Both kinds of stem cells are expected to be used in cell or gene therapies. The embryonic stem cell, however, has an ethical problem. The adult stem cell is actively researched to make nerve cells for therapeutic use, especially by using stem cells derived from bone marrow. The mesenchymal stem cell contained in the bone marrow is estimated to reach about 1×10⁶ cells of the total number so that it can be cultured from a small amount of the bone marrow collected. The method for culturing mesenchymal cells has been already established both home and abroad and applied to clinical trials. In practice, there was a report that mesenchymal stem can be used to treat patients of amyotropic lateral sclerosis. Hematopoietic stem cells and monocytes have been attempted to be injected into a spinal cavity. In addition, there is a report that autogenic mesenchymal stem cells were administered for the treatment of traumatic patients of spinal injury in domestic clinical tests. Further, according to a report from a clinical test currently under progress the mesencymal stem cells, when injected to severe patients of acroparalysis, showed partial improvement in motor neurons in wrists, fingers and elbows.

As described above, the cell therapy has been widely attempted to develop a method for the regeneration of a spinal cord injury. However, this cell technique is known to reduce the competency of nerve regeneration, because stem cells seldom last during a culture period. In order to overcome this problem, it is necessary to maintain the cell sources for a long period of time and to improve their competency. Therefore, various studies and efforts have been made to develop a most suitable tissue-engineered technology.

The tissue-engineered technique is conducted by using three components including a carrier, cells and physiologically active substances. In general, several kinds of tissue-engineered scaffolds have been prepared by combining a biocompatible and biodegradable carrier that increase the binding capacity of cells such as bone cells, tissue cells, cartilage cells and stem cells while surviving for a long period; and physiologically active materials that induce to proliferate and differentiate cells such as cytokines and growth factors.

Recently, many body organs are increasingly transplanted due to the advances in medical science, but organs donors are far below to meet the demand. As a consequence, to replace or regenerate a part of organ or tissue on injuries by the bio-engineered procedure, various kinds of biocompatible and biodegradable carriers have been developed actively.

In this bio-engineered procedure, it is essential to provide a support to reconstruct an adult stem cell with a 3-dimensional structure in an external environment which was isolated inside a body.

In the present invention, the biocompatible scaffolds for implanting cells are manufactured to regenerate a central nervous system.

The present inventors have adopted polyethylene glycol as an essential element that is a hydrophilic polymer, highly soluble in water and organic solvent, non-toxic and not rejected against immune reaction. The polyethylene glycol binds chemically with hydrophobic and biodegradable ester series polymers and increases the absorption of water in order to control the period of degradation when administered into a living body. In addition, ε-caprolactone (CL) as a hydrophobic and biodegradable ester series polymer is another essential element that can degrade and be secreted with biological metabolites after being dissolved, hydrolyzed chemically, degraded enzymatically and the like within a human body. The ε-caprolactone is also biocompatible and possibly adjusted in the molecular weight and chemical components in order to control the period of degradation. Besides, p-dioxanone (PDO), trimethylenecarbonate (TMC) or both PDO and TMC are polymerized in a particular ratio of content.

The phase transition of sol-gel according to temperature and concentration was studied by monitoring its hydrophilic parts and chemical structures. As a result, it was found that the sol-phase copolymers form gels at a temperature of human body when they are injected to rats.

The present inventors have developed the biocompatible and temperature-sensitive polyethylene glycol/biodegradable polyester block copolymer, which comprises a hydrophilic part comprising polyethylene glycol and a hydrophobic part comprising a biodegradable polyester containing ε-caprolactone (CL) segment as an essential element, a p-dioxanone (PDO) segment, a trimethylenecarbonate (TMC) segment or both segments of PDO and TMC, and has 2,000 to 7,000 g/mmol of molecular weight, and filed a patent application (Korean Patent Application No. 2006-0023991).

In addition, the present inventors have prepared the small intestinal submucosa tissue powder by treating the small intestinal submucosa with pepsin in an acidic solution, adjusting the pH of the mixture to be physiologically compatible followed by lyophilization. They have also developed an injectable hydrogel formed immediately after its injection into a living body. In this invention, the tissue powder was applied in a solution without using polymers (Korean Patent Application No. 2006-100430).

Based upon the above-mentioned methods, the present inventors have attempted to develop tissue-engineered scaffolds that combine adult stem cells and physiologically active materials in order to regenerate a central nervous system.

### SUMMARY OF THE DISCLOSURE

The present invention relates to tissue-engineered scaffolds that combine an adult stem cell or a nerve cell with a physiologically active material onto biocompatible carriers, wherein the biocompatible carrier is a biocompatible and temperature-sensitive polyethylene glycol/polyester block copolymer or a biocompatible hydrogel made of the tissue powder of a mucous membrane under small intestine capable of changing sol-gel phases.

Hence, the present invention is a modification of the above-mentioned patent applications. The object of the present invention is to provide tissue-engineered scaffolds that combine adult stem cells and physiologically active materials by using a biocompatible and temperature-sensitive polyethylene glycol/polyester block copolymer or biocompatible hydrogel made of small intestinal submucosa tissue powder with sol-gel phase transition behavior.

Another object of the present invention is to provide methods for regenerating nerve cells, wherein the tissue-engineered scaffold is implanted onto an acute or chronic injury of spinal cord after incising the injury partially or completely.

### BRIEF DESCRIPTION OF THE DRAWINGS

The above and other features of the present invention will now be described in detail with reference to certain exemplary embodiments thereof illustrated the accompanying drawings which are given hereinbelow by way of illustration only, and thus are not limitative of the present invention, and wherein:

FIG. 1 depicts the stem cells cultured in Example 1 and 2 that is observed under a phase-contrast microscope [(A) stem cells derived from muscle, (B) stem cells derived from fat, (C) stem cells derived from bone marrow, (D) stem cells derived from nerve, (E) olfactory ensheathing cells, (F) Schwann cells (40×)];

FIG. 2 depicts the phase transition of biocompatible hydrogel for the nerve regeneration, wherein (A) and (B) are copolymers prepared in Example 3, (A) shows a sol phase observed at 25° C. of room temperature, (B) shows a gel phase being changed at 37° C., (C) and (D) are the tissue powder of a mucous membrane under small intestine prepared in Example 4, (C) shows a sol phase observed at 25° C. of room temperature, and (D) shows a gel phase being changed at 37° C.;

FIG. 3 depicts the operation procedure conducted in Example 5, wherein (A) shows a feature of the operation, (B) shows spinal cord incised completely during the operation according to its length [(B1) 1 mm, (B2) 3 mm and (B3) 5 mm], (C) and (D) are the scaffolds of a tissue gel from the mucous membrane of small intestine in Example 4, and (E) and (F) are scaffolds of synthetic hydrogel described in Example 3];

FIG. 4 depicts the BBB scores evaluating the motion power of model in Example 6, wherein FIG. 4a shows a circular plate measuring motions, FIG. 4b shows a graph describing scores of injury model according to the length, and FIG. 4c shows a graph describing scores of each scaffold group;

FIG. 5 depicts the rehabilitation for improving the motion power, wherein FIG. 5a shows a motion of 1 mm model rotating on a cylinder, in which the hydrogel scaffold combining stem cells and growth factors in Example 3 is implanted and observed after 4 weeks, and FIG. 5b shows a swimming motion of the model;

FIG. 6 depicts the rehabilitation for improving the motion power, wherein FIG. 6a shows a motion of 1 mm model rotating on a cylinder, in which the tissue gel scaffold of the mucous membrane under small intestine combining stem cells and growth factors in Example 4 is implanted and observed after 4 weeks, and FIG. 6b shows a swimming motion of the model;

FIG. 7 depicts the motor evoked potential (MEP) test conducted in Example 7 for evaluating the recovery of nerves qualitatively;

FIG. 8 depicts the immunochemical staining conducted in Example 8 for evaluating the recovery of nerve cells histologically, wherein (A) shows a spinal cord after 4 weeks of implanting the hydrogel scaffold of Example 3, (B) shows a spinal cord after 4 weeks of implanting the gel scaffold of small intestinal submucosa in Example 4, (C1) shows an H & E of normal model, (C2) shows an NF of normal model, (C3) shows an NSE of normal model, (D1) shows an H & E of experimental model implanted with the hydrogel of Example 3, (D2) shows a NF of experimental model implanted with the hydrogel of Example 3, (D3) shows an NSE of an experimental model implanted with the hydrogel of Example 3, (D4) shows a GFAP of experimental model implanted with the hydrogel of Example 3, (D5) is a BrdU of experimental model implanted with the hydrogel of Example 3, (E1) shows an H & E of experimental model implanted with the tissue gel of small intestinal submucosa in Example 4, (E2) shows an NF of experimental model implanted with the tissue gel of small intestinal submucosa in Example 4, (E3) shows an NSE of experimental model implanted with the tissue gel of small intestinal submucosa in Example 4, (E4) shows a GFAP of experimental model implanted with the tissue gel of small intestinal submucosa in Example 4, and (E5) shows a BrdU of experimental model implanted with the tissue gel of small intestinal submucosa in Example 4.

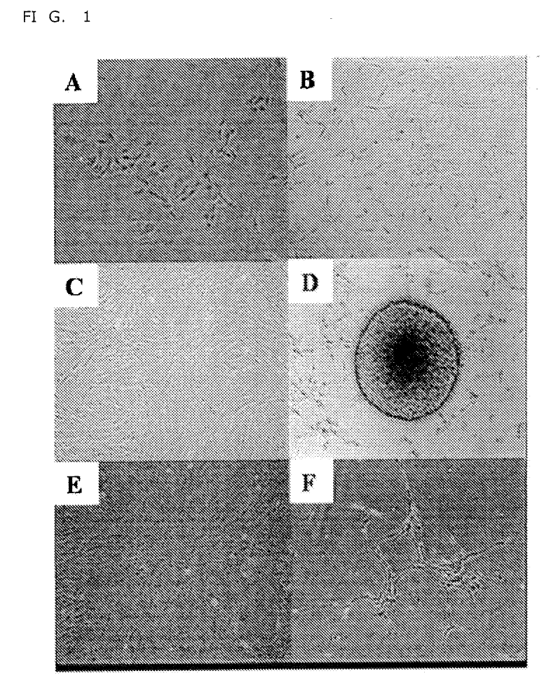

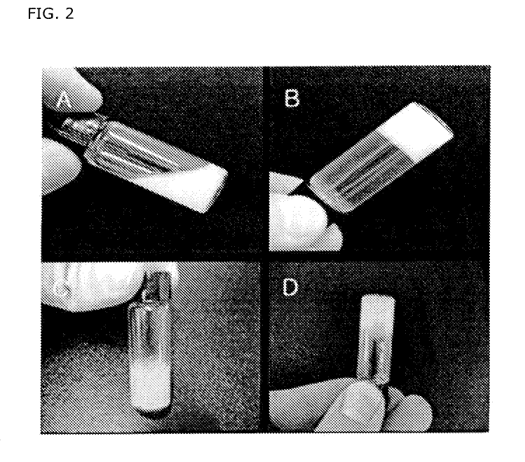

### BEST MODE FOR CARRYING OUT THE INVENTION

The present invention relates to a tissue-engineered scaffold which combine an adult stem cell or a nerve cell with a physiologically active material onto biocompatible carriers, wherein the biocompatible carrier is a biocompatible and temperature-sensitive polyethylene glycol/biodegradable polyester block copolymer or a biocompatible and injectable hydrogel made of tissue powder of a mucous membrane under small intestine with sol-gel phase transition behavior.

The biocompatible and temperature-sensitive polyethylene glycol/biodegradable polyester block copolymer comprises a hydrophilic part comprising polyethylene glycol and a hydrophobic part comprising biodegradable polyesters which contains a ε-caprolactone (CL) segment as an essential element and further, a p-dioxanone (PDO) segment, a trimethylenecarbonate (TMC) segment or segments of both PDO and TMC. The biocompatible and temperature-sensitive polyethylene glycol/biodegradable polyester block copolymer of the present invention has 2,000 to 7,000 g/mmol of molecular weight.

The polyethylene glycol contained in the hydrophilic part has 350 to 2,000 g/mol of molecular weight. The hydrophobic part comprises a compound of Formula I and each segment is polymerized randomly.

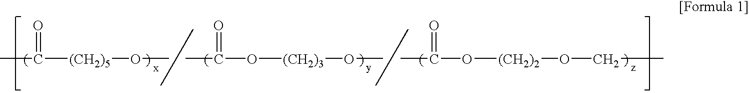

wherein x, y and z are segments independently forming a hydrophobic polyester part, wherein (x+y+z) is 100 mol %, y or z may be zero (0), x is 50 to 95 mol %, and (y+z) is 5 to 50 mol %.

The biocompatible small intestinal submucosa tissue powder with sol-gel phase transition behavior was prepared by treating the small intestinal submucosa with pepsin in an acidic solution, adjusting the pH of the resulting mixture to 5.5-7.8, followed by lyophilization. The copolymer or powder is prepared into an injectable hydrogel by adding a phosphate buffer to be used as a scaffold.

The present invention will be described in further details as set forth hereunder.

The present invention relates to a tissue-engineered scaffold capable of regenerating or recovering an injured spinal cord for central nervous system after being implanted to connect neurons, which combines adult stem cells or nerve cells and physiologically active materials on tissue-engineered carriers comprising biocompatible and temperature-sensitive polyethylene glycol/polyester block copolymer or biocompatible and injectable hydrogel made of small intestinal submucosa tissue powder with sol-gel phase transition behavior.

Above all, the adult stem cells and the nerve cells will be illustrated hereinafter.

The stem cell can replace any kind of tissues or cells correctly at any location and differentiate toward a precursor of specific cell. Thereby, the stem cell becomes an outstanding cell source useful for regenerative therapies and can treat and recover an injured organ effectively.

The mesenchymal stem cell derived from bone marrow has a complex feature of a stem cell group. In detail, this mesenchymal stem cell can be spreaded in vitro and differentiated into complex mesodermal cells, especially neurons. Thus obtained neuron is considered as an epoch to possibly treat neuronal diseases. The stem cell may be used to treat various neurotic diseases, if it is easily isolated and cultured in large scale. In addition, the stem cells derived from muscle and the stem cells derived from fat tissues have complex features of stem cell group to differentiate into various kinds of cells, depending on the cell environment. This stem cell can be separated easily and proliferate in vitro in a large scale. The nerve stem cell is so pluripotent as to differentiate toward blood, bone, cartilage and the like, regardless of germ layers. The nerve stem cell can be differentiated into neurons, gliacytes and the like to recover nerve tissues.

In addition, the olfactory ensheathing cellas a cell source of nervous system is a part of central nervous system and surrounds the olfactory nerve. This olfactory ensheathing cell is well known to play an important role to recover neurons. The Schwann cell is also regenerative for spinal cord and, in practice, can be used to perform autogenic implantation.

The procedure for culturing stem cells and nerve cells will be described clearly in the following Examples.

In the present invention, as the physiologically active material, a neutropin series nerve growth factor may be used, and is preferably selected from the group consisting of NGF (nerve growth factor), BDNF (brain-derived neurotropic factor), NT-3 (neurotrophin-3) and NT-4/5 (neurotrophin-4/5). It is a polypeptide as a soluble molecule that has a specific structure affecting the biological activities of cells. In detail, the physiologically active ingredient stimulates cell division to increase a cell number, changes cell shape and induces cell differentiation.

The procedure for manufacturing tissue-engineered scaffolds containing the stem cells and the growth factors will be described in further detail with the following Examples.

This scaffold plays an important role to isolate an impaired part of spinal cord and sustain cells and nerve generative inducers when nerve injuries are recovered. The tissue-engineered scaffold is often made of biodegradable synthetic polymers and natural substances. Recently, the mucous membrane tissue under small intestine is made of natural polymers of a living body solely or in a combination with synthetic polymers. In the present invention, the biocompatible carrier is made of temperature-sensitive polyethylene glycol/biodegradable polyester block copolymer and hydrogel made of the tissue powder of a mucous membrane of small intestine and prepared to the tissue-engineered scaffold.

Further features and advantages of the biocompatible and temperature-sensitive polyethylene glycol/biodegradable polyester block copolymer of the present invention will be explained more clearly hereinafter.

The polyethylene glycol (PEG) is used as a starting material to prepare the polyethylene glycol/biodegradable polyester block copolymer. PEG is advantageous to transfer drugs, since it easily adsorbs and excretes drugs as a vehicle. PEG has been already approved safe in a human body by FDA and used widely in pharmaceutical industries. It is highly soluble in water and organic solvents, non-toxic, not rejected against immune reaction and remarkably biocompatible. In addition, PEG restrains the adsorption of proteins most effectively among hydrophilic polymers and improves the biocompatibility of blood while being contacted. Therefore, it has been applied to a living body as a bio-material in various fields of industries. Unfortunately, this material containing PEG has a disadvantage that it is not biodegradeable. PEG has been known to increase the toxicity of cholesterol and neutral fat in blood plasma. To solve the above problem, low-molecular weight PEGs having less than 5,000 g/mol of molecular weight have been prepared, which can filtrate through kidney and be excreted easily from a living body. This PEG is polymerized with biodegradable ester series monomers that degrade to a biocompatible product through metabolism in human body to prepare the polyethylene glycol/biodegradable polyester block copolymer.

The biodegradable monomer of ester series is advantageous to control the period of degradation by adjusting its molecular weight and chemical composition. As a basic material, the block copolymer of polyethylene glycol (PEG) and polycaprolactone (PCL) has been applied to as a bio-material of a living body, since it is a temperature-sensitive copolymer changeable in sol-gel phases. However, the ε-caprolactone reduces the biocompatibility of tissues, is crystallized highly, and degrades slowly, although it is biodegradable and commercially available with various polymers.

As a consequence, the ε-caprolactone is polymerized with p-dioxanone (PDO), trimethylenecarbonate (TMC) or segments of both PDO and TMC in a particular ratio of content, in order to decrease the crystalline property and control the period of biodegradation. In detail, the ε-caprolactone is depicted in the following Formula 1, in which each segment forms a hydrophobic part randomly and irregularly.

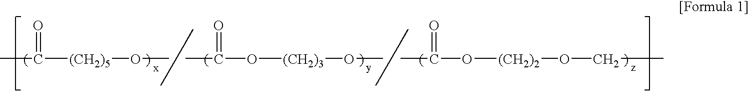

wherein x, y and z are segments independently forming a hydrophobic polyester part, wherein x is 50 to 95 mol % and (y+z) is 5 to 50 mol % (including y or z=0).

In the present invention, the low molecular weight (Mn=350 to 2,000 g/mole) polyethylene glycol forming a hydrophilic part and the ε-caprolactone (CL) of ester series are polymerized with p-dioxanone (PDO), trimethylenecarbonate (TMC) or both PDO and TMC. Particularly, the polyethylene glycol/polyester block copolymer of the present invention is prepared by the process: (1) azeotropic distilling polyethylene glycol and drying; (2) adding eater series monomers with methylene chloride (MC) or toluene as a reaction solvent and with an acidic catalyst as an activator of the monomers; and (3) polymerizing at −40 to 130° C. of a reaction temperature.

Preferably, the acidic catalyst can be one or more selected from a group comprising HCl, HBr, CF₃COOH, CCl₃COOH, BrCH₂COOH, CH₃COOH, BCl₃, BBr₃ and camphorsulfonic acid.

The polyethylene glycol/polyester block copolymer prepared above is a temperature-sensitive material showing the phase transition of sol-gel, which appears sol phase in a solution at room temperature and forms gel phase at 30 to 45° C. of temperature range and then, at 40 to 47° C. of temperature range more than a critical temperature, it returns to form sol phase. In this copolymer, the thermal property and the crystalline property are analyzed with a differential scanning calorimeter and an X-ray defractor. Also, the gel formation and the maintenance are observed when being injected into mice in order to examine its gel formation around at a body temperature.

This temperature-sensitive block copolymer should have a low viscosity, form gel rapidly and be excreted easily from a body due to a low molecular weight, when it is applied for injectable drug vehicles or functional carriers regenerating tissues. For this purpose, ester series p-dioxanone (PDO), trimethylenecarbonate (TMC) or both PDO and TMC are introduced in a particular ratio of content to the ε-caprolactone (CL) to reduce the crystalline property and decrease the viscosity. As a consequence, the copolymer has a preferable molecular weight and maintains a low viscosity and a low molecular weight satisfying the biocompatibility and the temperature-sensitivity of hydrogel.

Preferably, the polyethylene glycol/biodegradable polyester block copolymer has 2,000 to 7,000 g/mmol of molecular weight. When below 2,000 g/mmol of molecular weight, this copolymer continues to maintain a sol phase without sol-gel phase transition at the temperature range of a human body and dissatisfies the purpose of the invention. When above 7,000 g/mmol of molecular weight, this copolymer is slowly biodegraded due to a high molecular weight and needs a long period of recycle.

An embodiment of polyethylene glycol/biodegradable polyester block copolymer of the present invention is illustrated in the following Reaction Formula 1,

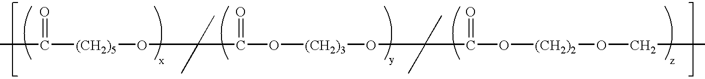

wherein n is an integer showing a repeating unit of polyethylene glycol forming a hydrophilic part; x, y and z are segments independently forming a hydrophobic polyester part; and x is 50 to 95 mole % and (y+z) is 5 to 50 mole % (including y or z=0).

This polyethylene glycol/biodegradable polyester block copolymer prepared above has a lower crystalline property than a basic model of polyethylene glycol-polycaprolactone block copolymer and thus has a lower viscosity to form gel phase rapidly. Besides, it is excreted easily from a body due to a low molecular weight, when it is applied for injectable drug vehicles or porous carriers regenerating tissues. Into the ε-caprolactone having the long period of biodegradation, p-dioxanone (PDO), trimethylenecarbonate (TMC) or both PDO and TMC are introduced in a particular ratio of the ε-caprolactone (CL) content to control the period. The solution of the polyethylene glycol/biodegradable polyester block copolymer can transit sol-gel phases at a wide range of temperature according to the biodegradable polymer including p-dioxanone (PDO) and/or trimethylcarbonate (TMC). This copolymer can be also used for a living body as bio-material, which becomes gel at a little lower or higher temperature than that of a living body as well as at the very body temperature.

In addition, the biocompatible tissue powder of a mucous membrane under small intestine can be applied for another carrier like drug vehicle of the present invention and will be explained clearly hereinafter.

When preparing the tissue powder, the mucous membrane of small intestine can be separated from mammals excluding human. Preferably, the mucous membrane under small intestine is separated from cows, pigs, rabbits and mice. This tissue is non-cellular tissue composed mostly of collagen and contains various growth factors, glucosaminoglycan, fibronectin and the like. Preferably, the growth factor can be transforming growth factor (TGF), insulin-like growth factor (IGF), acidic and basic fibroblast growth factor (aFGF and bFGF), vascular endothelial growth factor (VEGF), nerve growth factor (NGF) and the like. The glucosaminoglycan can be hyaluronic acid, chondroitin, chondroitin-4-sulfate, heparan sulfate, heparin, alginic acid, dextran, starch, chitin, chitosan, and the like.

The extracelluar substrate and the cytokines within the tissue of the mucous membrane under small intestine functions to attach, move, proliferate and grow cells. Also they are very useful in regenerating tissues or treat an injury and further, applicable to a drug vehicle.

Above all in order to prepare the tissue of the mucous membrane under small intestine of mammals, a mammal jejunum is separated from a small intestine, immersed in saline solution and phosphate buffer, treated to discard mesentery and then cut into pieces uniformly. The resulting small intestine is converted inside out and its outer layer is discarded by physical force. Then, the small intestine is converted again to remove serosa and muscle layers.

The mucous membrane of small intestine prepared above is lyophilized, sonicated, powdered and then, treated with an acidic solution and pepsin. This mixture solution is titrated at pH 5.5 to 7.8 and preferably at pH 6.5 to 7.5 in order to make gel phase of the biocompatible tissue powder.

The acidic solution can be any other solution adjusted at pH 2.5 to 4.5 and preferably, a solution containing 1 to 5 wt % of an acid selected among acetic acid, paratoluenesulfonic acid and maleic acid.

Preferably, the tissue of the mucous membrane under small intestine, the acidic solution and the pepsin are mixed in 1:1 to 5:0.1 to 2 of weight ratio. In this ratio range, the pepsin can loosen collagen chains of the tissue of the mucous membrane under small intestine effectively. This ratio gives a proper concentration and a proper condition.

The basic solution is used to titrate an acidic solution and to decrease an inflammatory reaction of adjacent cells during a subcutaneous injection or a tissue-engineered carrier. Preferably, the basic solution can be one or more selected from a group comprising NaOH, Na₂CO₃, NaHCO₃, Na₂HPO₄, Ca(HCO₃)₂, Ca(OH)₂, Ca(OH)NO₃, Ca(OH)Cl, Ca(OH)CN, KOH, NH₄OH and CH₃COONa. This basic solution is adjusted to pH 5.5 to 7.8 similar to that of a living body.

Without using polymers, the tissue powder of the mucous membrane under small intestine prepared above can form gel phase at the very site when being injected in a solution. It can be used for an injectable hydrogel excreting drug slowly.

The process for operating and regenerating an animal model of spinal cord injury will be described clearly hereinafter.

In the Examples of the present invention, mice were used as an experiment animal, but any mammal excluding human may be used.

In order to reduce the immune reaction of cells during recovery, Fischer rats were used as the animal model of spinal cord injury. The Fischer rats were impaired on central nervous system. First, the rats were cut their skin on the central longitudinal line of their backbone, removed of muscle membrane and muscles, T8 to T9 part of bones were exposed, and then, the exposed bones were removed. Here, it is essential to take an extreme caution to prevent a spinal cord injury. When exposed, the spinal cord was removed of its dura carefully with a micro surgical device and cut into pieces completely to 1 mm, 3 mm and 5 mm of size to establish a nerve injury model. Then, the tissue engineered scaffold that is combined with undifferentiated adult stem cells alone or in combination with the adult stem cells and olfactory ensheathing cells and Schwann cells and further physiologically active substances including growth factors, was implanted.

The experimental rats impaired in spinal cord after the operation should be cared carefully because they will be suffering from paraplegia. First, the rats were aerated with oxygen and rested on a heating bed at 37° C. to maintain their body temperature. Then, they were assisted to breathe smoothly without a stress, transferred to a new cage with fresh beddings and warmed. The rats were urinated twice a day and administered with antibiotics every day during a week. To help to improve their motional power (their physical status), they were allowed to exercise regularly and every day while being massaged, walking and swimming.

In the present invention, the recovery of neurons was estimated by conducting the evaluation of motional power, the motor evoked potential (MEP) and the histological examination as follows.

The evaluation of motional power was conducted by measuring BBB (Basso, Beattie, Bresnahan) scores and the data are graphed according to models. The motor evoked potential (MEP) was conducted to measure neurons quantitatively in a normal model or injured model of spinal cord and recovered model. The histological staining was performed to monitor the inflammatory reaction around an injury, the tissue formation and the regeneration of neurons. As a consequence, it was found that the central nerve can recover and regenerate after being implanted with the scaffold.

Therefore, based on the above-mentioned procedure, the present invention provides an improved method for recovering nerves in which the tissue-engineered scaffold combining regenerative cell source and physiologically active materials is used.

Practical and presently preferred embodiments of the present invention are illustrated as shown in the following Examples.

However, it will be appreciated that those skilled in the art, on consideration of this disclosure, may make modifications and improvements within the spirit and scope of the present invention. Especially, the biocompatible and temperature-sensitive polyethylene glycol/biodegradable polyester block copolymer or biocompatible and injectable hydrogel comprising tissue powder of a mucous membrane of small intestine changeable in sol-gel phase are already described in prior arts of Korean Patent Application Nos. 2006-23991 and 2006-100430. The disclosures in the above are incorporated to the present invention.

### EXAMPLE 1

### Isolation and Cultivation of Adult Stem Cells for Treating Injury of Spinal Nerve

Four kinds of adult stem cells for implantation on an injured part of spinal cord were separated as a cell source and cultured as follows.

As a first cell source, a stem cell derived from a skeletal muscle was isolated and differentiated into a nerve cell. In order to separate the stem cell derived from skeletal muscle, each femoral muscle region of 60 to 80 g of Fischer rats was cut to obtain muscles and then treated with collagenase to obtain single cells. The resulting cells were suspended with DMEM (Dulbecco's modified eagle medium) containing 5% fetal bovine serum, 5% horse serum and 2% antibiotics and seeded on a culture flask coated with collagen. In an hour, the cell supernatant was collected from the culture flask, centrifuged, washed by using fresh medium and again seeded on a culture flask. Most of fibroblasts were attached on the bottom of the culture flask within an hour. When being confluent in about 30 to 40%, the fibroblasts were collected from supernatant, centrifuged, washed by using a fresh medium and again seeded on a new culture flask. This procedure was repeated after 2 hours and 1, 2 and 3 days in order to separate stem cells derived from skeletal muscle. The resulting cells were seeded on a new cell flask at 10³ to 10⁴ cells/cm² of concentration and cultured at 37° C. and in 5% CO₂ incubator. The cultured cells were refreshed with a new medium every 3 days and subcultured every 15 days.

As a second cell source, a stem cell derived from fat was isolated and differentiated to a nerve cell. In order to separate the stem cell derived from fat, an abdominal fat region was cut from 60 to 80 g of Fischer rats and treated with collagenase to prepare single cells. The resulting cells were cultured with DMEM (Dulbecco's modified eagle medium) containing 10% fetal bovine serum and 1% antibiotics. The subculture was performed by the same procedure of the stem cell derived from skeletal muscle as described above.

As the third cell source, a stem cell derived from a bone marrow was isolated and differentiated to a nerve cell. In order to separate the stem cell derived from the bone marrow, each femur and tibia of 60 to 80 g of Fischer rats was cut and run through the bone cavity by using a 1 ml syringe and phosphate buffered saline to collect cells. The resulting cells were centrifuged and purified. The stem cells were cultured with DMEM (Dulbecco's modified eagle medium) containing 10% fetal bovine serum and 1% antibiotics. The subculture was performed by the same procedure of the stem cell derived from skeletal muscle as described above.

As the fourth cell source, a nerve stem cell was isolated and differentiated to a nerve cell. In order to separate the nerve stem cell, each skull obtained from 60 to 80 g of Fischer rats was opened to isolate a brain part purely. The brain tissue was cut into pieces with a scissor, washed twice and cultured. The subculture was performed by the same procedure of the stem cell derived from skeletal muscle as described above.

### EXAMPLE 2

### Isolation and Cultivation of Nerve Cells for Treating Injury of Spinal Nerve

An olfactory ensheathing cell and a Schwann cell were separated as a cell source and cultured to regenerate an injured part of spinal cord as follows.

As a first cell source, the olfactory ensheathing cell was separated from each skull of 60 to 80 g Fischer rats. Each skull was operated to isolate an olfactory organ, washed with HBSS buffer twice and then, treated with 0.125% trypsin to prepare single cells. The resulting cells were cultured with F12/DMEM (Dulbecco's modified eagle medium) containing 10% fetal bovine serum and subcultured every 15 days while being refreshed with a new medium every 3 days.

As a second cell source, the Schwann cell was separated from 60 to 80 g Fischer rats. Sciatic nerve were isolated and immersed in Leibovitiz-15 medium to remove epithelial cells. The resulting nerve was cut into 1 mm size of pieces and cultured with DMEM media containing 10% fetal bovine serum. Then, the Schwann cells were cultured for 5 weeks while being refreshed with a new medium twice a week. The Schwann cells were transferred to a new flask every week.

### EXAMPLE 3

### Preparation of Polyethylene Glycol/Biodegradable Polyester Block Copolymer

In order to prepare MPEG-PCL/PPDO block copolymers having 3,150 g/mole of molecular weight, 1.67 g (2.24 mmole) of methoxypolyethylene glycol (MPEG) as an initiator and 80 mL of toluene were mixed in a 100 mL round flask under a well-dried state and distilled for 3 hours at 130° C. by using a Dean stock trap. After being distilled, the toluene remnant was discarded completely and the resulting methoxypolyethylene glycol (MPEG) was cooled to a room temperature. Then, 5.08 g (44.5 mmol) of ε-caprolactone (CL) and 0.28 g (2.62 mmol) of p-dioxanone (PDO) were injected in purified forms and then, 25 mL of methylene chloride (MC) was added after purified with a reaction solvent. Afterward, 4.5 mL of HCl was injected as a polymerizing catalyst and stirred for 24 hours at room temperature. The total process was performed with high-purity nitrogen. In order to remove non-reactive monomers or raw material after the reaction, 600 mL of hexane and 150 mL of heptane were used to precipitate the reactant while being dropwisely added. The resulting precipitate was dissolved with methylene chloride and filtered through a filter paper. After the solvent was removed with a rotary evaporator, the precipitate was dried under a reduced pressure. The block copolymer prepared above was sterilized with EO gas, dissolved in 15 to 20 wt % of phosphate buffered saline and then dispersed homogeneously to manufacture an injectable gel.

### EXAMPLE 4

### Preparation of Biocompatible Tissue Gel of a Mucous Membrane Under Small Intestine

A porcine jejunum was collected within 4 hours of slaughter, and washed cleanly with flowing water and cut into pieces uniformly. The outer layers of the jejunum including serosa, dense layer and mucous membrane of muscle were discarded under physical force of a pincer. Then, the resultant was washed completely to prepare tube-shaped tissues of a mucous membrane of small intestine and stored at −80° C. with an ultra-low refrigerator before use.

The mucous membrane of small intestine prepared above was lyophilized, sonicated with a mixer, and then powdered to 10 to 30 μm of size with a mill. 1 wt % of the resulting powder was mixed 3 wt % acetic acid, 0.1 wt % pepsin and 3′DW and then, stirred for 48 hours. The resulting solution of a mucous membrane of small intestine was adjusted to pH 7.4 by using Na₂CO₃, NaHCO₃ and NaOH. The optimized solution was lyophilized and pulverized with a mill. As a result, the biocompatible tissue powder of a mucous membrane of small intestine was manufactured. This final powder was identified to form an injectable gel, when adding a phosphate buffered saline (pH 7.4).

### EXAMPLE 5

### Implantation of Scaffold Combining Stem Cell and Physiological Substances

The scaffolds for regenerating nerves prepared in the above Examples were implanted to experimental animals as follows.

The gel for regenerating nerves prepared in the Example 3 and 4 was combined with the stem cell prepared in the Example 1 and 2 solely or with the stem cell, the olfactory ensheathing cell and the Schwann cell in an appropriate ratio while being adjusted in 2×10⁶ of total cell number. Then, BDNF (200 ng/scaffold) as a nerve growth factor was added to make a scaffold for an implant.

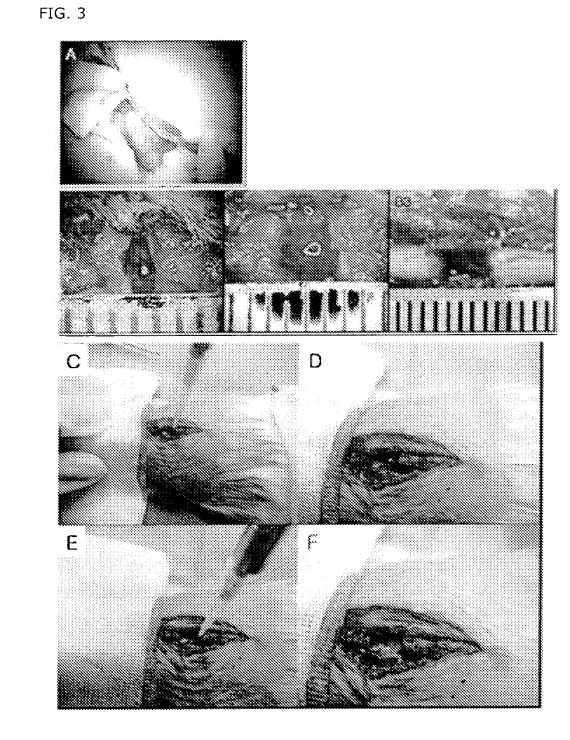

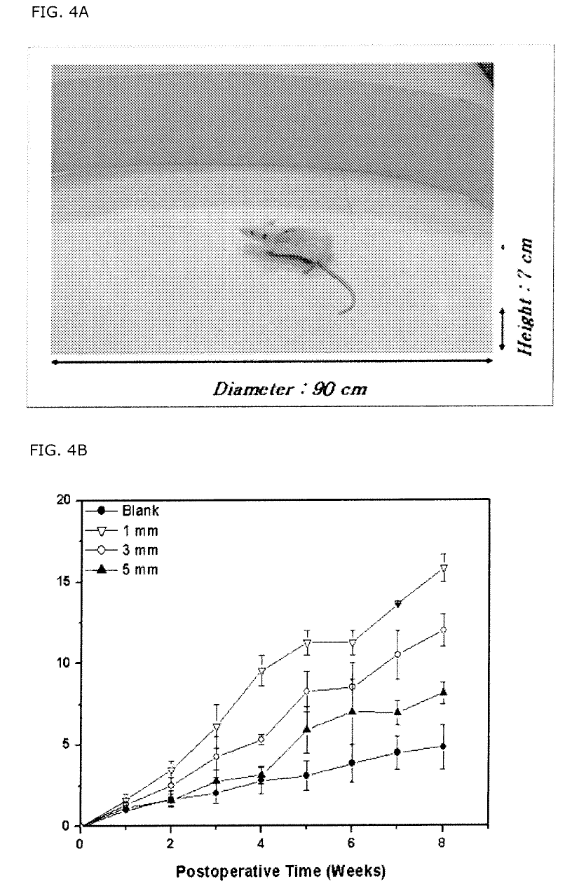

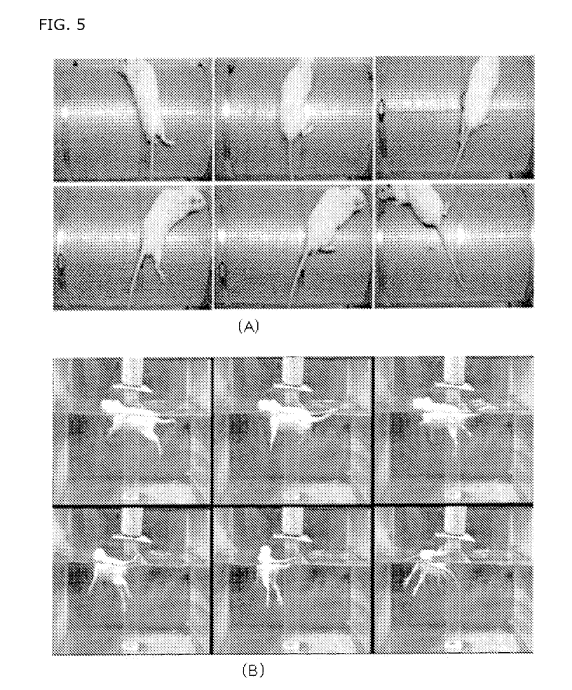

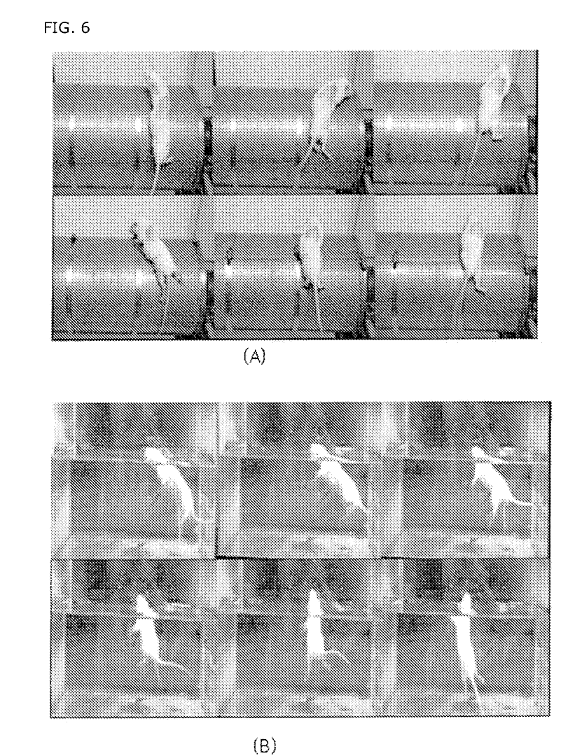

Fischer female rats aged 5-weeks with 80 to 100 g of body weight were operated to injure their thoracic vertebrate part (T8˜T9) and cut into pieces in each size as described in FIG. 5. Then, the tissue-engineered scaffold was implanted. After the operation, the experimental rats were aerated with oxygen and rested on a heating bed at 37° C. until coming round from narcosis. Then, they were treated to pass urine twice a day and administered with antibiotics every day during a week (See FIG. 3).

### EXAMPLE 6

### Evaluation of Regeneration by Improving Motional Power

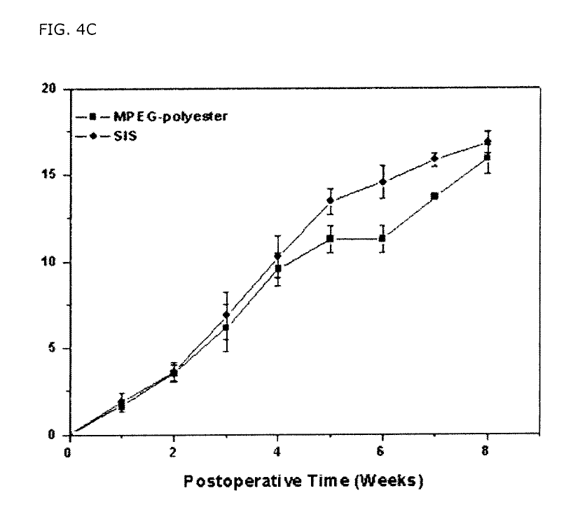

In order to increase the motional power of a spinal injury model, the experimental rats were massaged every day after one week of the operation and strengthened in muscle power by using a rotary chamber. Also, they were exercised to swim to improve motions of hind legs. In order to evaluate the motional power of models, BBB (Basso, Beattie, Bresnahan) scores were measured every week after the operation (See FIG. 4). Before the evaluation, the rats were exercised on a rotary chamber and then, observed the movement on a rotary plate. Scores were determined according to BBB scale to measure the motion. The motion of hind limbs, foot shapes, foot angle, body and the like, were considered for the scores. As a result, it was observed that the motional power should be higher in the experimental group implanted with the scaffold than blank group without a scaffold. The motion became more active when less injured. In addition, especially in 1 mm model, the natural tissue gel of a mucous membrane under small intestine increased the motion power more or less higher than the synthetic hydrogel in each group. Consequently, it was confirmed that both the experimental group improved the motion effectively to regenerate a central nervous system.

### EXAMPLE 7

### Evaluation of Regeneration by Motor Evoked Potential (MEP)

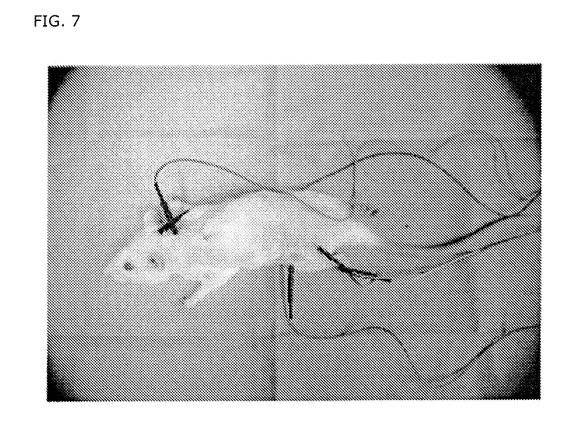

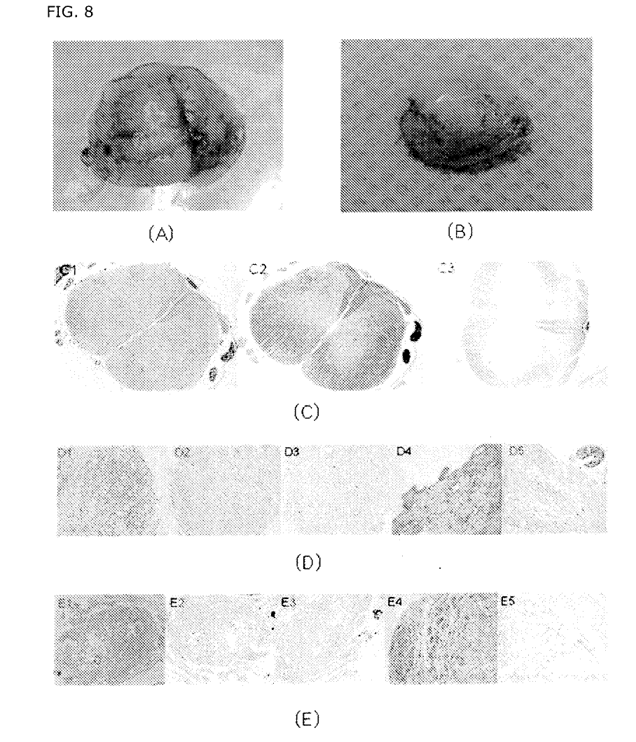

In order to estimate the recovery of spinal nerves, a motor evoked potential (MEP) was measured as follows (See FIG. 7). The motor evoked potential (MEP) records minute electric action potentials generated when muscles contract during a motion. The motor evoked potential (MEP) is often used to monitor a pathway of movement and measures potentials in muscle, peripheral nerve or spinal cord, when stimulating motor cortex of cerebrum or a motor pathway of central nervous system. The motor evoked potential tests were conducted repeatedly 4 weeks and 8 weeks after the operation to measure latency (m/s) and amplitude (pV). This result was analyzed to observe the regeneration of neurons. As a consequence, it is identified that the animal model implanted with a scaffold for nerve regeneration can recover more than a control group without a scaffold in about 30 to 40%.

TABLE 1

| items | nerve/sites | latency (m/s) | amplitude (μV) |
|---|---|---|---|
| cutting model | fore limb | 1.10 | 27041 |
| | hind limb | 6.10 | 738.3 |
| 1 mm model | fore limb | 0.90 | 16910 |
| | hind limb | 4.28 | 408.4 |
| 3 mm model | fore limb | 1.55 | 10321 |
| | hind limb | 6.03 | 238.3 |

TABLE 2

| | | | amplitude |
|---|---|---|---|
| scaffolds | nerve/sites | latency (m/s) | (μV) |
| Scaffold of Example 3 | hind limb | 5.10 | 313.5 |
| Scaffold of Example 3 | hind limb | 4.60 | 418.3 |

### EXAMPLE 8

### Histological Examination

In order to examine the nerve regeneration histologically, animal tissues were delivered after 4 weeks and 8 weeks of the operation. The resulting tissues were cut vertically to measure the number of neurons and stained with H & E to judge whether causing inflammation between carrier and cell-implanted part, whether forming a new tissue or whether neurons growing well. As a result, it was confirmed that the inflammatory reaction is not found around a lesion after implanting a carrier and cells. Also, a new tissue was made around the spinal cord nerve. In order to identify the recovery of nerve, immunohistochemical staining was performed by using specific markers for nerve cells such as NF (neurofilament; SCYTEK, USA), NSE (neuron-specific enolase; Serotec, UK) and GFAP (glial fibrillary acidic protein; Dako, Denmark) to evaluate the regeneration. In addition, the stem cells were labeled with Brdu (bromodeoxyuridine; Dako, Denmark) before being implanted and chased to examine the effects on cell survival and regeneration. Consequently, it is elucidated that the stem cells should survive and induce the nerve regeneration around an injured spinal nerve when observing the growth of neurons and gliacytes.

### INDUSTRIAL APPLICABILITY

As illustrated and confirmed above, the present invention provides a tissue-engineered scaffold combining nerve regenerative cell source and physiologically active materials, which can regenerate or recover an injured spinal nerve to connect neurons after being implanted. In the present invention, the biocompatible and biodegradable carriers can improve the attachment of various cells such as nerve regenerative cells, bone cells, tissue cells and cartilage cells and survive the cells for a long time. They are also combined with various cytokines or growth factors affecting the cell growth and differentiation to manufacture the tissue-engineered scaffolds. Therefore, the present invention will contribute to improve the capacity to recover nerves. This tissue-engineered scaffold can be used to replace a part of impaired organs or tissues due to the lack of organ donors. It can be manufactured in various 3-dimensional structures to regenerate or replace irregularly impaired organs or tissues. Further, the biocompatible and biodegradable scaffolds are more convenient and useful for, when it is prepared into an injectable form.

Those skilled in the art will appreciate that the conceptions and specific embodiments disclosed in the foregoing description may be readily utilized as a basis for modifying or designing other embodiments for carrying out the same purposes of the present invention.

Those skilled in the art will also appreciate that such equivalent embodiments do not depart from the spirit and scope of the invention as set forth in the appended claims.

1. A tissue-engineered scaffold comprising a combination of at least one adult stem cell and a physiologically active material on a biocompatible carrier, wherein the biocompatible carrier is a biocompatible and temperature-sensitive polyethylene glycol/biodegradable polyester block copolymer or a biocompatible and injectable hydrogel made of small intestinal submucosa tissue powder with sol-gel phase transition behavior.

2. The tissue-engineered scaffold of claim 1, wherein the biocompatible and temperature-sensitive polyethylene glycol/biodegradable polyester block copolymer has 2,000 to 7,000 g/mmol of molecular weight, wherein a hydrophilic part thereof comprises polyethylene, glycol and a hydrophobic part thereof comprises a biodegradable polyesters comprising ε-caprolactone (CL) segment as an essential element, a p-dioxanone (PDO) segment, a trimethylenecarbonate (TMC) segment or segments of both PDO and TMC.

3. The tissue-engineered scaffold of claim 2, wherein the polyethylene glycol contained in the hydrophilic part has 350 to 2,000 g/mol of molecular weight.

4. The tissue-engineered scaffold of claim 2, wherein the hydrophobic part comprises a compound of formula 1 and each segment is polymerized randomly. 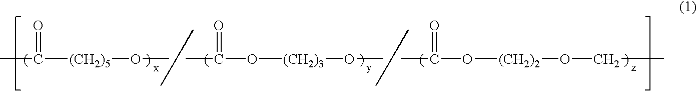
wherein x, y and z are segments independently forming a hydrophobic polyester part, wherein (x+y+z) is 100 mol %, y or z may be zero (0), x is 50 to 95 mol %, and (y+z) is 5 to 50 mol %.

5. The tissue-engineered scaffold of claim 1, wherein the biocompatible small intestinal submucosa tissue powder with sol-gel phase transition behavior is prepared by treating small intestinal submucosa tissue with pepsin in an acidic solution, adjusting the pH of the resulting mixture to 5.5-7.8; and lyophilization.

6. The tissue-engineered scaffold of claim 1, wherein the injectable hydrogel is prepared by combining to the biocompatible and temperature-sensitive polyethylene glycol/biodegradable polyester block copolymer or the biocompatible tissue powder of a mucous membrane of small intestine changeable in sol-gel phases with phosphate buffered saline; and gellated.

7. The tissue-engineered scaffold of claim 1, comprising a plurality of adult stem cells.

8. The tissue-engineered scaffold of claim 1, wherein the at least one adult stem cell is obtained from muscle, fat, nerve, bone marrow, olfactory ensheathing cells or Schwann cells.

9. The tissue-engineered scaffold of claim 8, wherein the at least are adult stem cell is obtained from bone marrow, and is a mesenchymal stem cell.

10. The tissue-engineered scaffold of claim 8, which is prepared by combining at least one adult stem cell with a physiologically active material on the biocompatible carrier.

11. The tissue-engineered scaffold of claim 5, wherein the pH is adjusted to from pH 6.5 to 7.5.

12. A method for regenerating nerve cells, which comprises implanting the tissue-engineered scaffold of claim 1, onto an acute or chronic injury of a spinal cord after incising the injury partially or completely.

13. The method of claim 12, wherein the tissue-engineered scaffold is implanted in injectable form.

14. The method of claim 12, which further compares massaging the acute or chronic injury beginning one week after the implanting.
